(19) United States
(12) Patent Application Publication
(10) Pub. No.: US 2021/0202055 A1
(43) Pub. Date: Jul. 1, 2021

(54) Learning Interpretable Strategies in the Presence of Existing Domain Knowledge

(71) Applicant: International Business Machines Corporation, Armonk, NY (US)

(72) Inventors: Cao Xiao, Cambridge, MA (US); Zachary Shahn, Brooklyn, NY (US); Daby M. Sow, Croton on Hudson, NY (US); Mohamed Ghalwash, Yorktown Heights, NY (US); Sanjoy Dey, White Plains, NY (US)

(21) Appl. No.: 16/730,107

(22) Filed: Dec. 30, 2019

(51) Int. Cl. G16H 20/00; G16H 50/30; G16H 10/60; G06N 20/00; G06F 17/18

(52) U.S. Cl. CPC G16H 20/00; G16H 50/30; G06F 17/18; G06N 20/00; G16H 10/60

(57) ABSTRACT

A mechanism computes a discounted health variable with a penalty for deviating from clinical guidelines based on a distance function representing an allowed deviation from the clinical guidelines, applies reinforcement learning techniques on the discounted health variable to generate a reinforcement learning (RL) model for generating dynamic treatment regimes, and determines, for a patient for a plurality of times, a next action in a treatment regime using the RL model with no distance function, an optimal next action in the treatment regime with allowed deviation from the guidelines, and a next action in the treatment regime that adheres to the guidelines. The mechanism generates an outcome output display based on the determined next action in a treatment regime using the RL model with no distance function, optimal next action in the treatment regime with allowed deviation from the guidelines, and next action in the treatment regime that adheres to the guidelines.

### BACKGROUND

The present application relates generally to an improved data processing apparatus and method and more specifically to mechanisms for learning interpretable treatment strategies in the presence of existing domain knowledge.

An electronic health record (EHR) is the systematized collection of patient and population electronically-stored health information in a digital format. These records can be shared across different health care settings. Records are shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems are designed to store data accurately and may be used to capture the state of a patient across time. It eliminates the need to track down a patient's previous paper health records and assists in ensuring data is accurate and legible. Due to the digital information being searchable and in a single file, EHRs are more effective when extracting health data for the examination of possible trends and long-term changes in a patient. Population-based studies of health records may also be facilitated by the widespread adoption of EHRs.

Like any number of industries, healthcare is being transformed by the explosion of low-cost data. In healthcare, the transformation is driven in large part by instrumentation of healthcare institutions, instrumentation of patients outside of healthcare institutions, EHR adoption and general digitization. There have been many benefits. End users can take advantage of quantities of newly available information to solve problems in population health, clinical decision support, and patient engagement, among other applications.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising a processor and a memory, the memory comprising instructions that are executed by the processor to specifically configure the processor to implement a dynamic treatment regime generation engine for learning interpretable treatment strategies in the presence of existing domain knowledge. The method comprises computing optimal dynamic treatment strategies or regimes by optimizing a health outcome variable discounted for any measurable deviations from clinical knowledge obtained from existing clinical guidelines or best practices among other sources. The method further comprises applying reinforcement learning techniques on the discounted health variable to generate a reinforcement learning (RL) model for generating dynamic treatment regimes. The method further comprises determining, for a patient for a plurality of times, a next action in a treatment regime using the RL model with no restrictions with respect to prior knowledge, an optimal next action in the treatment regime taking into account deviations from prior knowledge, and a next action in the treatment regime that strictly adheres to the guidelines, thus providing a natural way for end user to interpret and ground the dynamic treatment regimes generated by the system within their existing frame of mind represented here by the prior knowledge that could be encapsulated in best practices and clinical guidelines. The method further comprises generating an outcome output display based on the determined next action in a treatment regime using the RL model with no restrictions with respect to prior knowledge, optimal next action in the treatment regime taking into account with allowed deviations from prior knowledge, and next action in the treatment regime that adheres to the prior knowledge. The method further comprises presenting the outcome output to a user.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive healthcare system in a computer network;

FIG. 2 is a block diagram of an example data processing system in which aspects of the illustrative embodiments are implemented;

FIG. 3 is an example diagram illustrating an interaction of elements of a healthcare decision support system in accordance with one illustrative embodiment;

FIG. 4 is a block diagram of an interpretable strategy generator in accordance with an illustrative embodiment;

FIG. 5 is a block diagram of a model builder for learning interpretable strategies in the presence of existing domain knowledge in accordance with an illustrative embodiment;

FIG. 6 illustrates an example presentation layer for a treatment regime in accordance with an illustrative embodiment;

FIG. 7 illustrates an example presentation layer depicting the impact of different treatment regimes in accordance with an illustrative embodiment;

FIG. 8 is a flowchart illustrating operation of a mechanism for building a reinforcement learning model to dynamically generate treatment regimes with adherence to guidelines in accordance with an illustrative embodiment;

FIG. 9 is a flowchart illustrating operation of a mechanism for reinforcement learning with adherence to guidelines in accordance with an illustrative embodiment; and

FIG. 10 is a block diagram illustrating operation of a mechanism for dynamically generating treat regimes with adherence to clinical guidelines and best practices in accordance with an illustrative embodiment.

### DETAILED DESCRIPTION

One major drawback of data driven approaches that learn predictive models is that they may not be interpretable. Building deep learning networks from observational data can produce powerful mathematical functions that accurately predict phenomena of interest. However, the complexity of these models may reduce their usefulness as domain experts often experienced difficulties to map these complex functions into their existing knowledge and mental models. This problem is further exacerbated when computer programs are not only asked to make predictions but also tasked to make decisions or provide treatment recommendations.

Learning under knowledge constraints remains an open research area. Learning models that are explainable or interpretable according to current state of the art knowledge in a domain is unsolved. In healthcare, AI models are often required to be interpretable for end users to trust and consume the output of these models. However, the concept of interpretability is ill defined. The illustrative embodiment formalizes interpretability as a deviation from the mental frame of the actor receiving the output of the AI model. In other words, AI model outputs that are in concordance with the mental frame of mind of the actor are deemed more interpretable than AI model outputs that do not map well into the mental frame of mind of the actor. To formalize this approach in a healthcare setting, we formalize the mental frame of mind of the actor (e.g., a clinician) with models based on best practices and clinical guidelines. Deviations of AI model outputs from guidelines or best practices are estimated to measure the degree of interpretability and are represented in front of the actor for proper decision support. In an RL setting, guidelines are treated in this invention as “soft” constrained and handled by decision support systems in a way that mimics how physicians interpret and use guidelines.

Machine learning models fall broadly into two main categories:

transparent interpretable models exposing the rationale behind their computations to the external world (including humans) and black box models with learned
computations that are opaque to the external world. In healthcare, the former set of models are often preferable. However, despite recent arguments for models that fall solely in this set, the use of opaque models is quite prevalent given the complexity of the tasks that are solved by these models. While some researchers advocate for more interpretable AI models, the illustrative embodiments are focused on interpretability from an end user perspective by developing a system able to put any AI model output within the context of existing end user domain knowledge. The illustrative embodiments focus solely on AI models recommending actions, thus corresponding to the Reinforcement Learning (RL) branch of Machine Learning, within AI.

The illustrative embodiments provide techniques that restrict the data driven search for RL models to interpretable models that can be explained by distances between model outputs and existing knowledge that can be represented by guidelines or best practice models. Consequently, while we do not attempt to interpret the AI model itself, the illustrative embodiments provide a natural way to interpret model outputs with explanations that are relevant for domain experts. The illustrative embodiments restrict this class of model outputs to tree structures that refer to observable quantities that are meaningful to domain experts. This restriction enforces a language based on trees used by the proposed system to explain its outputs. More complex higher-level languages could be considered including natural languages forcing the AI model to produce explanation of its outputs in a more complex form tailored for the end users. The gist of the illustrative embodiments is to jointly learn both model for the generation of dynamic treatment regimes together with models mapping the generated dynamic treatment regimes with existing knowledge. The illustrative embodiments also estimate penalties during optimization in the form of regularizing terms that penalize discrepancies between the outputted dynamic treatment regimes and the existing knowledge. The illustrative embodiments penalize the complex model for its deviations from accepted clinical guidelines and best practices as a way to ensure that one can project back its recommendations into existing and accepted domain knowledge.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general-purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine-readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

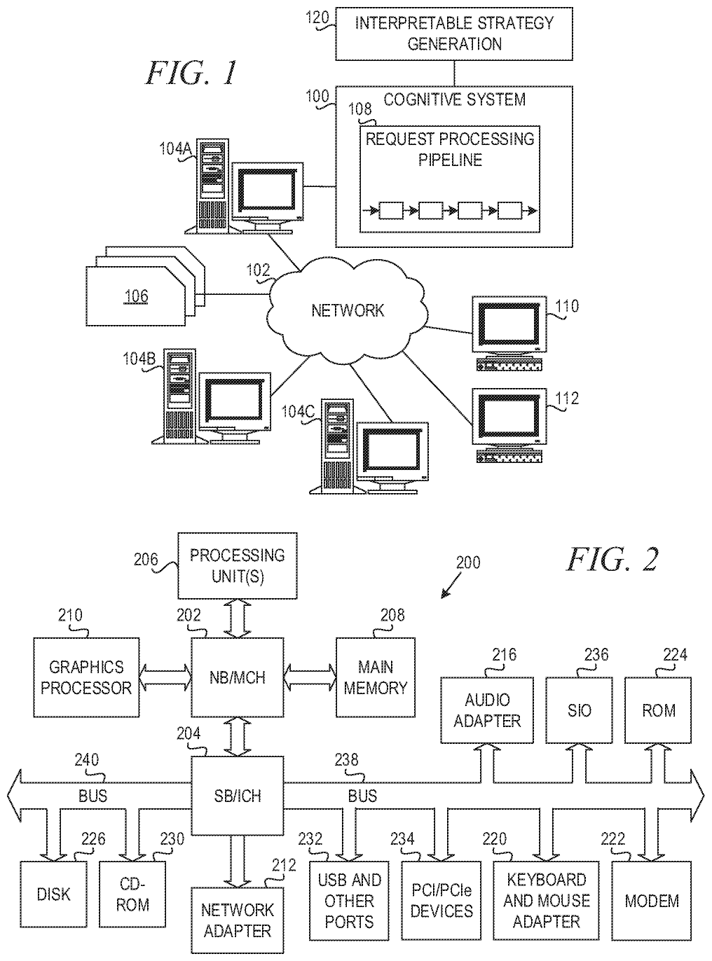

As noted above, the present invention provides mechanisms for processing health care clinical data controlled datasets. The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for healthcare applications (also referred to herein as a “healthcare cognitive system”) which implements a request processing pipeline, request processing methodology, and request processing computer program product with which the mechanisms of the illustrative embodiments are implemented. These requests may be provided as structured or unstructured request messages, or any other suitable format for requesting an operation to be performed by the healthcare cognitive system. As described in more detail hereafter, the particular healthcare application that is implemented in the cognitive system of the present invention is a healthcare application for presenting relevant information using a graphical presentation engine.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to a first medical malady domain (e.g., various types of cardiovascular diseases) while another request processing pipeline may be trained to answer input requests in another medical malady domain (e.g., various types of cancers). In other cases, for example, the request processing pipelines may be configured to provide different types of cognitive functions or support different types of healthcare applications, such as one request processing pipeline being used for patient diagnosis, another request processing pipeline being configured for cognitive analysis of EHR data, another request processing pipeline being configured for patient monitoring, etc.

Moreover, each request processing pipeline may have its own associated datasets that it ingests and operates on, e.g., one dataset for cardiovascular disease domain documents and another dataset for cancer diagnostics domain related documents in the above examples. These datasets may include, but are not limited to, EHR data and other historical patient data.

As will be discussed in greater detail hereafter, the illustrative embodiments may be integrated in, augment, and extend the functionality of these mechanisms of a healthcare cognitive system with regard to learning interpretable strategies in the presence of existing domain knowledge. Thus, it is important to first have an understanding of how cognitive systems are implemented before describing how the mechanisms of the illustrative embodiments are integrated in and augment such cognitive systems and request processing pipeline mechanisms. It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

FIG. 1 depicts a schematic diagram of one illustrative embodiment of a cognitive system 100 implementing a request processing pipeline 108 in a computer network 102. The cognitive system 100 is implemented on one or more computing devices 104A-C (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-C. The network 102 includes multiple computing devices 104A-C, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 may provide cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language question, natural language request for information, natural language request for the performance of a cognitive operation, or the like, and the answer may be returned in a natural language format maximized for efficient comprehension in a point-of-care clinical setting. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-C on the network 102 include access points for content creators and cognitive system users. Some of the computing devices 104A-C include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and input requests to the cognitive system 100 that are answered/processed based on the content in the corpus or corpora of data 106. In one embodiment, the requests are formed using natural language. The cognitive system 100 parses and interprets the request via a pipeline 108, and provides a response to the cognitive system user, e.g., cognitive system user 110, containing one or more response to the request, results of processing the request, or the like. In some embodiments, the cognitive system 100 provides a response to users in a ranked list of candidate answers/responses while in other illustrative embodiments, the cognitive system 100 provides a single final answer/response or a combination of a final answer/response and ranked listing of other candidate answers/responses.

The cognitive system 100 implements the pipeline 108, which comprises a plurality of stages for processing an input request based on information obtained from the corpus or corpora of data 106. The pipeline 108 generates answers/responses for the input request based on the processing of the input request and the corpus or corpora of data 106.

In the context of the present invention, cognitive system 100 may provide a cognitive functionality for assisting with healthcare-based operations. For example, depending upon the particular implementation, the healthcare based operations may comprise patient diagnostics medical practice management systems, personal patient care plan generation and monitoring, patient electronic medical record (EHR) evaluation for various purposes, such as for identifying patients that are suitable for a medical trial or a particular type of medical treatment, or the like. Thus, the cognitive system 100 may be a healthcare cognitive system 100 that operates in the medical or healthcare domains and which may process requests for such healthcare operations via the request processing pipeline 108 input as either structured or unstructured requests, or the like.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for interpretable strategy generation engine 120 for learning interpretable strategies in the presence of existing domain knowledge. The approach of the illustrative embodiment is to bias the search for optimal strategies towards plans that conform to existing guidelines by introducing regularizers that penalize deviations from guidelines and that also penalize models that are not conformed with current practices.

Interpretability has received a good amount of attention for machine learning classification problems. These surrogate approaches have been applied in deep learning where interpretable surrogate models are learned separately and often sequentially in a post-hoc way (e.g., after a deep model is learned).

This illustrative embodiment differs from this prior art as it provides a principled way to provide explanations in Reinforcement Learning (RL) settings (as opposed to classification settings) where the AI is tasked to infer optimal action plans or treatment strategies for the optimization of a well-defined outcome. We propose to learn interpretable dynamic treatment strategies by regularizing the training of an RL model with measures of distances in the action space between actions from existing domain knowledge (such as current practices or treatment guidelines) and the outputs of the RL model.

The intuition behind the joint training is to strike a balance between a black-box deep model and interpretability. By regularizing the optimization and forcing the learning towards well established practices according to prior knowledge, the learned RL model becomes more prone to produce hypotheses that are interpretable to humans since humans tend to understand such prior knowledge.

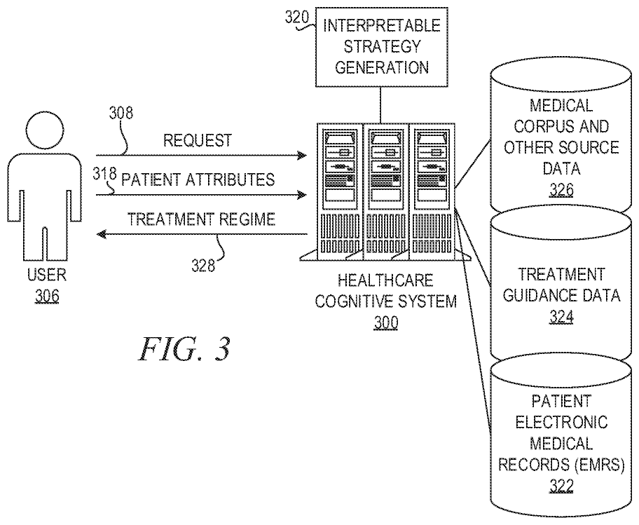

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which implements a cognitive system 100 augmented to include the additional mechanisms of the illustrative embodiments described hereafter.

In the depicted example, data processing system 200 employs a hub architecture including North Bridge and Memory Controller Hub (NB/MCH) 202 and South Bridge and Input/Output (I/O) Controller Hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

FIG. 3 is an example diagram illustrating an interaction of elements of a healthcare decision support system in accordance with one illustrative embodiment. The example diagram of FIG. 3 depicts an implementation of a healthcare decision support system 300 that is configured to provide a summary of EHR data for patients. However, it should be appreciated that this is only an example implementation and other healthcare operations may be implemented in other embodiments of the healthcare decision support system 300 without departing from the spirit and scope of the present invention.

Moreover, it should be appreciated that while FIG. 3 depicts the user 306 as a human figure, the interactions with user 306 may be performed using computing devices, medical equipment, and/or the like, such that user 306 may in fact be a computing device, e.g., a client computing device. For example, interactions between the user 306 and the healthcare decision support system 300 will be electronic via a user computing device (not shown), such as a client computing device 110 or 112 in FIG. 1, communicating with the healthcare decision support system 300 via one or more data communication links and potentially one or more data networks.

As shown in FIG. 3, in accordance with one illustrative embodiment, the user 306 submits a request 308 to the healthcare decision support system 300, such as via a user interface on a client computing device that is configured to allow users to submit requests to the healthcare decision support system 300 in a format that the healthcare decision support system 300 can parse and process. The request 308 may include, or be accompanied with, information identifying patient attributes 318. These patient attributes 318 may include, for example, an identifier of the patient 302, social history, and demographic information about the patient, symptoms, and other pertinent information obtained from responses to requests or information obtained from medical equipment used to monitor or gather data about the condition of the patient. In one embodiment, patient attributes 318 may include identification of a biomedical image for processing to detect anomalies. Any information about the patient that may be relevant to a cognitive evaluation of the patient by the healthcare cognitive system 300 may be included in the request 308 and/or patient attributes 318.

The healthcare decision support system 300 provides an AI system that is specifically configured to perform an implementation specific healthcare oriented cognitive operation. In the depicted example, this healthcare oriented cognitive operation is directed to providing a summary of EHR data 322 to the user 306 to assist the user 306 in treating the patient based on their reported symptoms and other information gathered about the patient. The healthcare decision support system 300 operates on the request 308 and patient attributes 318 utilizing information gathered from the medical corpus and other source data 326, treatment guidance data 324, and the patient EHRs 322 associated with the patient to generate treatment regime 328. In one embodiment, patient EHR data 322 may include biomedical images. In accordance with the illustrative embodiments, a treatment regime 328 is dynamically generated based on historical patient date, such as patient attributes 318 and EHR data 322, and clinical guidelines, which may be incorporated into medical corpus and other source data 326. The treatment regime 328 may be presented in a ranked ordering with associated supporting evidence, obtained from the patient attributes 318 and data sources 322-326, indicating the reasoning as to why portions of EHR data 322 are being provided.

In accordance with the illustrative embodiments herein, the healthcare cognitive system 300 is augmented to include interpretable strategy generation engine 320 for dynamically generating treatment regimes 328 in which soft constraints obtained from guidelines and best practices are imposed while learning a reinforcement learning (RL) model. Interpretable strategy generation engine 320 imposes concordance to guidelines and best practices constraints during the construction (or learning) of treatment strategies. Interpretable strategy generation engine 320 allows the user to understand the impact of conforming to or deviating from guidelines while following dynamically generated treatment regime 328.

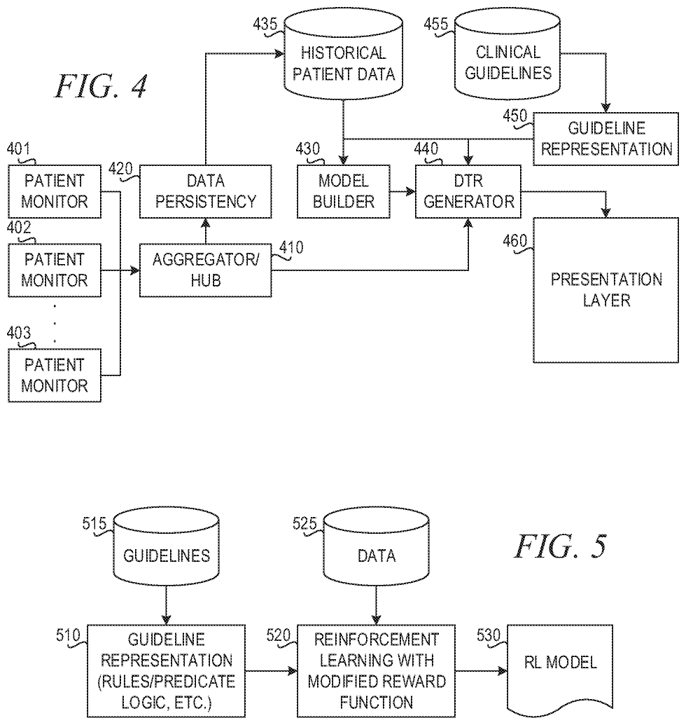

FIG. 4 is a block diagram of an interpretable strategy generator in accordance with an illustrative embodiment. Aggregator/hub 410 aggregates patient data from patient monitors 401, 402, 403, and data persistency component 420 persists the patient data into historical patient data store 435. Clinical guidelines store 455 contains guidelines including guidelines provided by doctors and current practices, which may be learned from data. Guidelines and current practices may be specific to a given hospital, a given institution, or a given doctor.

Model builder 430 uses historical patient data 435 to learn a model for dynamically generating treatment regimes. Dynamic treatment regimes are essentially a set of actions recommended by the RL model. In this embodiment, we represent them as a tree with nodes consisting of various tests applied on the covariates collected from the patients. For example, in a critical care environment, a dynamic treatment regime produced by the RL model may suggest that “if the arterial blood pressure of the patient falls below some precomputed thresholds, the patient may switch treatment from fluid resuscitation to vasopressors.” In accordance with the illustrative embodiment, a guideline representation 450 is formed from clinical guidelines 455. In this embodiment, this guideline representation is performed a priori by transforming clinical guidelines into logical assertions allowing the system to test whether a set of actions included within a dynamic treatment regime are in accordance with the clinical guidelines. Furthermore, the same approach is adopted to test whether parts of a dynamic treatment regime are in accordance with existing practices. In another embodiment one skilled in the art may define more general distances between dynamic treatment regimes and guidelines or existing practices. Consequently, the model builder 430 imposes guidelines 450 and interpretability constraints during construction of treatment strategies based on historical patient data 435 using reinforcement learning.

Dynamic treatment regime (DTR) generator 440 then uses the model built by model builder 430 to a generate treatment regimes based on historical patient data 435, guideline representation 450, and patient data received from aggregator/hub 410. The DTR generator 440 provides the generated treatment regime to presentation layer 460, which presents the treatment regime to a patient or doctor. In accordance with the illustrative embodiment, presentation layer 460 allows the user to understand the impact of conforming to or deviating from guidelines 450 while following the dynamically generated treatment regime.

FIG. 5 is a block diagram of a model builder for learning interpretable strategies in the presence of existing domain knowledge in accordance with an illustrative embodiment. Guideline representation 510 is formed from guidelines 515. Guideline representation 510 may use rules, predicate logic, etc. The illustrative embodiment uses reinforcement learning with a modified reward function 520 using data 525 as training data to form reinforcement learning model 530. The illustrative embodiment proposes changing the reward structure in reinforcement learning to penalize deviations from the guidelines 510.

To learn a model for generating treatment regimes, the reinforcement learning computes a probability of survival as follows:

Q_{K}(L̅_{K},Ā_{K})=E[Y*|L̅_{K},Ā_{K}]

where:
a. L̅_{K} represents the history of all observed data for a patient up to time k
b. Ā_{K} represents the history of all past actions that where taken on a given patient up to time k
c. Y* denote the optimal outcome variable that the RL is optimizing
d. Q_{K} is the Q function as defined in the Q-learning algorithm in RL that estimates the average treatment effect.

The reinforcement learning then uses the following equation to find the action that minimizes Q_{K};

V_{K}(L̅_{K},Ā_{K-1})=maxₐ_{_{K}}Q_{K}(L̅_{K},(Ā_{K-1},a_{K}))

where:
a. V_{K} is the value function optimizing Q_{K} under the action a_{K} taken at time k

The reinforcement learning then computes for a health outcome variable Y a discounted health outcome variable Y* as follows:

$${Y}^{*} = Y - \frac{λ}{K} {\sum }_{m - 0}^{K} {δ}_{m} ( {A}_{m} , {a}_{m}^{*} ( {\overline{L}}_{m} , {\overline{A}}_{m - 1} ) ) ,$$

where δₘ represents a distance function that measures deviation from existing knowledge (e.g., guidelines and prior knowledge); L̅ₘ represents again a history of covariates up to time m, while Āₘ₋₁ represents actions in the treatment regimen up to time m−1. The illustrative embodiment applies reinforcement learning techniques estimating Y*, a discounted reward that is being optimized. 2 is a real number representing a hyperparameter controlling how much impact the distance function δₘ has on optimization. Large values of λ impose stricter prior knowledge adherence constraints.

Computing m can be done in several ways. At each time m, δₘ evaluates a distance between prior knowledge and the optimal action Aₘ. We represent this prior knowledge as a set of possible actions aₘ*. Using the covariate and action histories, aₘ* returns all admissible actions according to this prior knowledge. With aₘ* defined, there are many ways to compute δₘ. In our preferred embodiment, δₘ is simply a Hamming distance, estimated to be 0 if aₘ*(L̅ₘ,Āₘ₋₁) contains Aₘ and 1 otherwise. Hence, the quantity

$$\frac{1}{K} {\sum }_{m - 0}^{K} {δ}_{m} ( {A}_{m} , {a}_{m}^{*} ( {\overline{L}}_{m} , {\overline{A}}_{m - 1} ) ) \leq 1$$

can be interpreted as a measure of how much the RL is deviating from prior knowledge and optimizing for Y* takes these deviations into account.

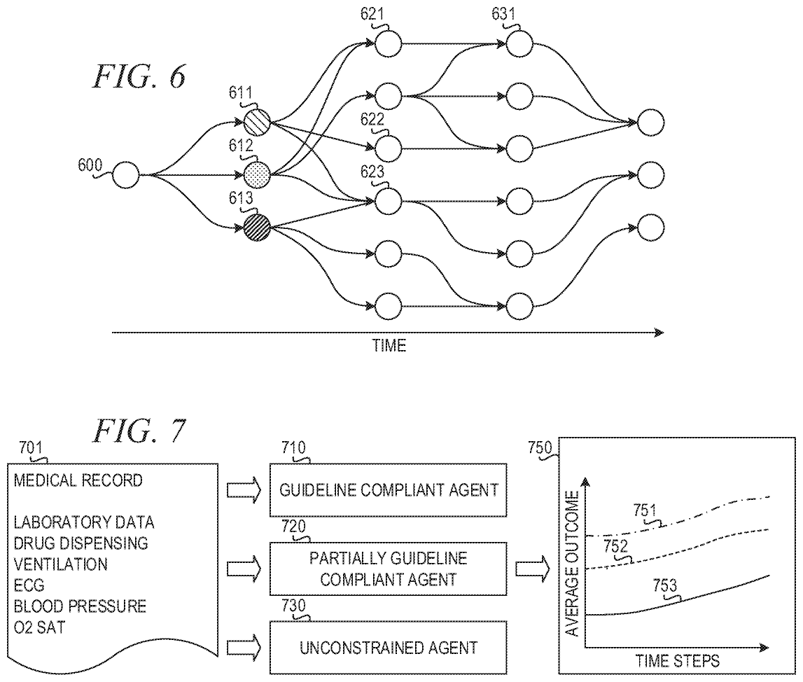

FIG. 6 illustrates an example presentation layer for a treatment regime in accordance with an illustrative embodiment. At each point in time, the presentation layer identifies a next action (ARN) in the treatment regime with no imposed constraints from the distance function δₘ on the corresponding estimated discounted health outcome variable. (AROP) identifies an optimal next action in the treatment regime using the distance function with the corresponding estimated discounted health outcome variable assuming optimality, and (ARG) identifies the best next action that adheres to the guidelines with the corresponding estimated discounted health outcome variable assuming optimality.

Thus, from a given action 601, the presentation layer identifies ARN 611, AROP 612, and ARG 613. In this example, ARN 611, AROP 612, and ARG 613 are different actions. From action 611, the presentation layer identifies ARN 621, AROP 622, and ARG 623. Again, these are different actions. Thus, the presentation layer allows the user to see how actions deviate from the guidelines, where ARG 613 and ARG 623 are actions that conform fully to the guidelines. This embodiment assumes a pre-existing metric within the action space, thus allowing comparisons between ARN, AROP and ARG for rendering within the presentation layer. The distance δₘ described above can be used to compare ARG with AROP and ARN.

From action 621, the presentation layer identifies ARN, AROP, and ARG, which are the same action 631. Thus, the presentation layer reduces the tree. The presentation layer also collapses identical actions in the tree. For example, both action 611 and action 612 lead to action 621.

Other trees may be generated from the presentation layer and overlay on top of one another to “explain” to the user the impact of his/her actions (e.g., computing the discounted health outcome variable when all actions are strictly adhering to the guidelines). The presentation layer allows the user to explicitly visualize the adherence or deviation of the treatment regime from the guideline when hovering on a node of the tree.

FIG. 7 illustrates an example presentation layer depicting the impact of different treatment regimes in accordance with an illustrative embodiment. FIG. 7 illustrates how a user may perceive the impact of different treatment regimes on the average outcome Y. In this figure, different values for λ are used by each of the agents shown in 710, 720, and 730 based on medical record 701. For agent 710, λ is set to be very large (theoretically infinite and very large in practice), thus preventing the agent from selecting strategies that are not in accordance with guidelines. For agent 730, λ is set to 0, thus allowing the agent to focus on the optimization of Y irrespective to deviations from guidelines or prior knowledge. Agent 720 represents the intermediate case where λ is non-zero and of moderated value. With λ, the end user is able to control how much concordance with existing knowledge should be enforced in the learning of treatment strategies by the agent.

In the example depicted in FIG. 7, the output 750 illustrates the average output Y generated by the agents 710, 720, 730. This output 750 presents curve 751 generated by agent 710, curve 752 generated by agent 720, and curve 753 generated by agent 730. Thus, a user may discern the impact of different treatment regimes based on different values for λ. In accordance with the illustrative embodiment, output 750 allows the user to understand the impact of conforming to or deviating from guidelines while following the dynamically generated treatment regime.

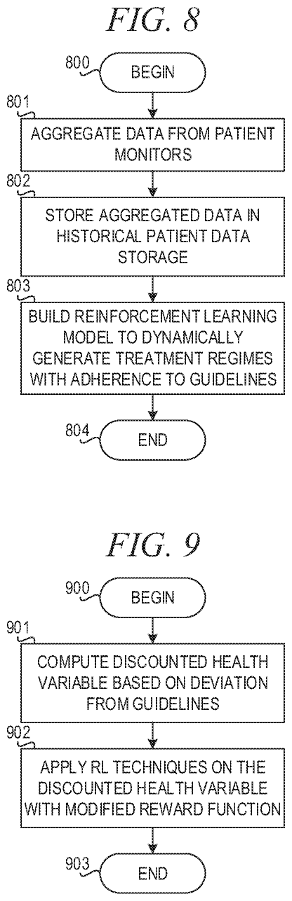

FIG. 8 is a flowchart illustrating operation of a mechanism for building a reinforcement learning model to dynamically generate treatment regimes with adherence to guidelines in accordance with an illustrative embodiment. Operation begins (block 800), and the mechanism aggregates data from patient monitors (block 801). The mechanism stores the aggregated data in historical patient data storage (block 802). Then, the mechanism builds a reinforcement learning model to dynamically generate treatment regimes with adherence to guidelines (block 803). Thereafter, operation ends (block 804).

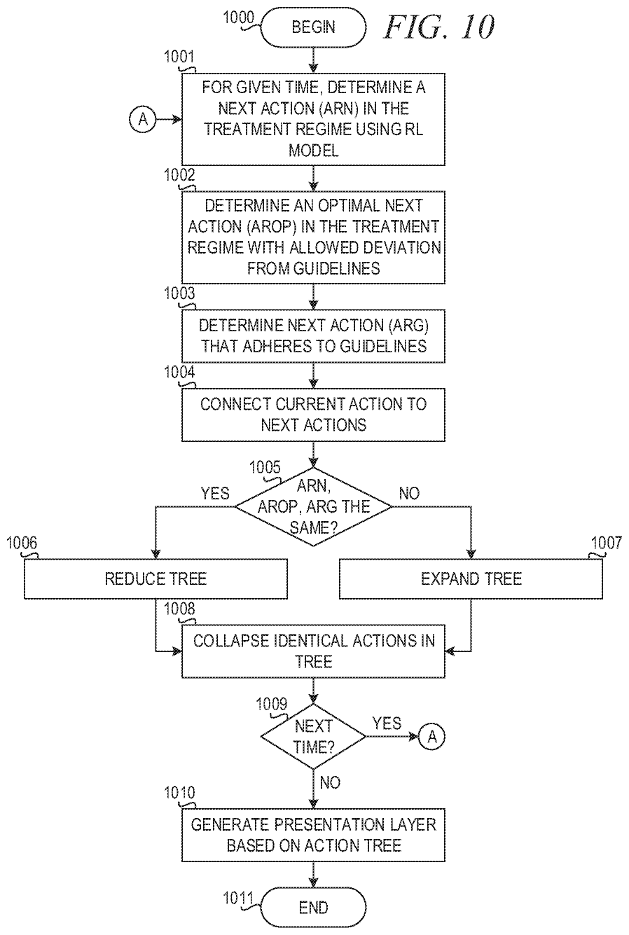

FIG. 9 is a flowchart illustrating operation of a mechanism for reinforcement learning with adherence to guidelines in accordance with an illustrative embodiment. Operation begins (block 900), and the mechanism computes a discounted health variable with a penalty for deviating from the guidelines (block 901). The mechanism applies reinforcement learning techniques on the discounted health variable with modified reward function based on deviation from the guidelines (block 902). Thereafter, operation ends (block 903).

FIG. 10 is a block diagram illustrating operation of a mechanism for dynamically generating treat regimes with adherence to clinical guidelines and best practices in accordance with an illustrative embodiment. Operation begins (block 1000), and for each given time, the mechanism determines a next action (ARN) in the treatment regime using the reinforcement learning model (block 1001), determines an optimal next action (AROP) in the treatment regime with allowed deviation from the guidelines (block 1002), and determines a next action (ARG) that adheres to guidelines (block 1003). The mechanism then connects the current action to the next actions (block 1004).

The mechanism determines whether ARN, AROP, and/or ARG are the same (block 1005). If any two or more of ARN, AROP, and ARG are the same, then the mechanism reduces the tree (block 1006); otherwise, the mechanism expands the tree (block 1007). Then, the mechanism collapses identical actions in the tree (block 1008).

The mechanism then determines whether to consider the next time (block 1009). If the mechanism determines to consider the next time, then operation returns to block 1001; otherwise, the mechanism generates the presentation layer based on the action tree (block 1010) and operation ends (block 1011).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication-based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions that are executed by the processor to specifically configure the processor to implement a dynamic treatment regime generation engine for learning interpretable strategies in the presence of existing domain knowledge, the method comprising:
computing a discounted health variable with a penalty for deviating from clinical guidelines and/or best practices based on a distance function representing an allowed deviation from the clinical guidelines and/or best practices;
applying, by a model builder executing within the data processing system, reinforcement learning techniques on the discounted health variable to generate a reinforcement learning (RL) model for generating dynamic treatment regimes;
determining, by the dynamic treatment regime generation engine, for a patient for a plurality of times, an unconstrained next action in a treatment regime using the RL model with no constraint, a partially guideline compliant next action in the treatment regime with allowed deviation from the guidelines, and a guideline compliant next action in the treatment regime that adheres to the guidelines;
generating, by a presentation layer within the dynamic treatment regime generation engine, an outcome output display based on the determined next action in a treatment regime using the RL model with no constraint, optimal next action in the treatment regime with allowed deviation from the guidelines, and next action in the treatment regime that adheres to the guidelines; and
presenting, by the presentation layer, the outcome output display to a user.

2. The method of claim 1, wherein applying reinforcement learning techniques on the discounted health variable comprises:
computing an average outcome value as follows:
Q_{K}(L̅_{K},Ā_{K})=E[Y*|L̅_{K},Ā_{K}]
where:
L̅_{K} represents a history of all observed data for a patient up to time k;
Ā_{K} represents the history of all past actions that where taken on a given patient up to time k;
Y* denote the optimal outcome variable that the RL is optimizing; and
Q_{K} is a Q function as defined in a Q-learning algorithm in RL that estimates the average treatment effect.

3. The method of claim 2, wherein applying reinforcement learning techniques on the discounted health variable further comprises:
using the following equation to find an action that minimizes Q_{K};
V_{K}(L̅_{K},Ā_{K-1})=maxₐ_{_{K}}Q_{K}(L̅_{K},(Ā_{K-1},a_{K}))
where:
V_{K} is the value function optimizing Q_{K} under the action a_{K} taken at time k; and
computing for a health outcome variable Y a discounted health outcome variable Y* as follows:
$${Y}^{*} = Y - \frac{λ}{K} {\sum }_{m - 0}^{K} {δ}_{m} ( {A}_{m} , {a}_{m}^{*} ( {\overline{L}}_{m} , {\overline{A}}_{m - 1} ) ) ,$$
where δₘ represents a distance function that measures deviation from existing domain knowledge; L̅ₘ represents a history of covariates up to time m; Āₘ₋₁ represents actions in the treatment regimen up to time m−1; aₘ* represents all admissible actions according to the prior domain knowledge; and λ is a real number representing a hyperparameter controlling how much impact the distance function δₘ has on optimization.

4. The method of claim 3, wherein δₘ is a Hamming distance, estimated to be 0 if aₘ*(L̅ₘ,Āₘ₋₁) contains Aₘ and 1 otherwise.

5. The method of claim 1, wherein applying reinforcement learning techniques on the discounted health variable comprises aggregating data from patient monitors and storing aggregated data in a historical patient data storage.

6. The method of claim 1, generating the outcome output display comprises generating an action tree based on the determined next action in a treatment regime using the RL model with no distance function, optimal next action in the treatment regime with allowed deviation from the guidelines, and next action in the treatment regime that adheres to the guidelines.

7. The method of claim 6, wherein generating the action tree comprises:
connecting current actions to next actions;
responsive to two or more of the determined next action in a treatment regime using the RL model with no distance function, the optimal next action in the treatment regime with allowed deviation from the guidelines, and the next action in the treatment regime that adheres to the guidelines being the same, reducing the action tree; and
responsive to the determined next action in a treatment regime using the RL model with no distance function, the optimal next action in the treatment regime with allowed deviation from the guidelines, and the next action in the treatment regime that adheres to the guidelines not being the same, expanding the action tree.

8. A computer program product comprising a computer readable storage medium having a computer readable program stored therein, wherein the computer readable program, when executed on a data processing system, causes the data processing system to implement a dynamic treatment regime generation engine for learning interpretable strategies in the presence of existing domain knowledge, wherein the computer readable program causes the data processing system to:
compute a discounted health variable with a penalty for deviating from clinical guidelines based on a distance function representing an allowed deviation from the clinical guidelines;
apply, by a model builder executing within the data processing system, reinforcement learning techniques on the discounted health variable to generate a reinforcement learning (RL) model for generating dynamic treatment regimes;
determine, by the dynamic treatment regime generation engine, for a patient for a plurality of times, an unconstrained next action in a treatment regime using the RL model with no distance function, a partially guideline compliant next action in the treatment regime with allowed deviation from the guidelines, and a guideline compliant next action in the treatment regime that adheres to the guidelines;
generate, by a presentation layer within the dynamic treatment regime generation engine, an outcome output display based on the determined next action in a treatment regime using the RL model with no distance function, optimal next action in the treatment regime with allowed deviation from the guidelines, and next action in the treatment regime that adheres to the guidelines; and
present, by the presentation layer, the outcome output display to a user.

9. The computer program product of claim 8, wherein applying reinforcement learning techniques on the discounted health variable comprises:
computing a probability of survival as follows:
Q_{K}(L̅_{K},Ā_{K})=E[Y*|L̅_{K},Ā_{K}]
where:
L̅_{K} represents a history of all observed data for a patient up to time k;
Ā_{K} represents the history of all past actions that where taken on a given patient up to time k;
Y* denote the optimal outcome variable that the RL is optimizing; and
Q_{K} is a Q function as defined in a Q-learning algorithm in RL that estimates the average treatment effect.

10. The computer program product of claim 9, wherein applying reinforcement learning techniques on the discounted health variable further comprises:
using the following equation to find an action that minimizes Q_{K};
V_{K}(L̅_{K},Ā_{K-1})=maxₐ_{_{K}}Q_{K}(L̅_{K},(Ā_{K-1},a_{K}))
where:
V_{K} is the value function optimizing Q_{K} under the action a_{K} taken at time k; and
computing for a health outcome variable Y a discounted health outcome variable Y* as follows:
$${Y}^{*} = Y - \frac{λ}{K} {\sum }_{m - 0}^{K} {δ}_{m} ( {A}_{m} , {a}_{m}^{*} ( {\overline{L}}_{m} , {\overline{A}}_{m - 1} ) ) ,$$
where δₘ represents a distance function that measures deviation from existing domain knowledge; L̅ₘ represents a history of covariates up to time m; Āₘ₋₁ represents actions in the treatment regimen up to time m−1; aₘ* returns all admissible actions according to this prior knowledge; and λ is a real number representing a hyperparameter controlling how much impact the distance function δₘ has on optimization.

11. The computer program product of claim 10, wherein δₘ is a Hamming distance, estimated to be 0 if aₘ*(L̅ₘ,Āₘ₋₁) contains Aₘ and 1 otherwise.

12. The computer program product of claim 8, wherein applying reinforcement learning techniques on the discounted health variable comprises aggregating data from patient monitors and storing aggregated data in a historical patient data storage.

13. The computer program product of claim 8, generating the outcome output display comprises generating an action tree based on the determined next action in a treatment regime using the RL model with no distance function, optimal next action in the treatment regime with allowed deviation from the guidelines, and next action in the treatment regime that adheres to the guidelines.

14. The computer program product of claim 13, wherein generating the action tree comprises:
connecting current actions to next actions;
responsive to two or more of the determined next action in a treatment regime using the RL model with no distance function, the optimal next action in the treatment regime with allowed deviation from the guidelines, and the next action in the treatment regime that adheres to the guidelines being the same, reducing the action tree; and
responsive to the determined next action in a treatment regime using the RL model with no distance function, the optimal next action in the treatment regime with allowed deviation from the guidelines, and the next action in the treatment regime that adheres to the guidelines not being the same, expanding the action tree.

15. A data processing system comprising:
at least one processor; and
at least one memory coupled to the at least one processor, wherein the at least one memory comprises instructions which, when executed by the at least one processor, cause the at least one processor to implement a dynamic treatment regime generation engine for learning interpretable strategies in the presence of existing domain knowledge, wherein the instructions cause the processor to:
compute a discounted health variable with a penalty for deviating from clinical guidelines based on a distance function representing an allowed deviation from the clinical guidelines;
apply, by a model builder executing within the data processing system, reinforcement learning techniques on the discounted health variable to generate a reinforcement learning (RL) model for generating dynamic treatment regimes;
determine, by the dynamic treatment regime generation engine, for a patient for a plurality of times, an unconstrained next action in a treatment regime using the RL model with no distance function, a partially guideline compliant next action in the treatment regime with allowed deviation from the guidelines, and a guideline compliant next action in the treatment regime that adheres to the guidelines;
generate, by a presentation layer within the dynamic treatment regime generation engine, an outcome output display based on the determined next action in a treatment regime using the RL model with no distance function, optimal next action in the treatment regime with allowed deviation from the guidelines, and next action in the treatment regime that adheres to the guidelines; and
present, by the presentation layer, the outcome output display to a user.

16. The data processing system of claim 15, wherein applying reinforcement learning techniques on the discounted health variable comprises:
computing a probability of survival as follows:
Q_{K}(L̅_{K},Ā_{K})=E[Y*|L̅_{K},Ā_{K}]
where:
L̅_{K} represents a history of all observed data for a patient up to time k;
Ā_{K} represents the history of all past actions that where taken on a given patient up to time k;
Y* denote the optimal outcome variable that the RL is optimizing; and
Q_{K} is a Q function as defined in a Q-learning algorithm in RL that estimates the average treatment effect.

17. The data processing system of claim 16, wherein applying reinforcement learning techniques on the discounted health variable further comprises:
using the following equation to find an action that minimizes Q_{K};
V_{K}(L̅_{K},Ā_{K-1})=maxₐ_{_{K}}Q_{K}(L̅_{K},(Ā_{K-1},a_{K}))
where:
V_{K} is the value function optimizing Q_{K} under the action a_{K} taken at time k; and
computing for a health outcome variable Y a discounted health outcome variable Y* as follows:
$${Y}^{*} = Y - \frac{λ}{K} {\sum }_{m - 0}^{K} {δ}_{m} ( {A}_{m} , {a}_{m}^{*} ( {\overline{L}}_{m} , {\overline{A}}_{m - 1} ) ) ,$$
where δᵢₙ represents a distance function that measures deviation from existing domain knowledge; L̅ₘ represents a history of covariates up to time m; Āₘ₋₁ represents actions in the treatment regimen up to time m−1; aₘ* returns all admissible actions according to this prior knowledge; and λ is a real number representing a hyperparameter controlling how much impact the distance function δₘ has on optimization.

18. The data processing system of claim 17, wherein δₘ is a Hamming distance, estimated to be 0 if aₘ*(L̅ₘ,Āₘ₋₁) contains Aₘ and 1 otherwise.

19. The data processing system of claim 15, wherein applying reinforcement learning techniques on the discounted health variable comprises aggregating data from patient monitors and storing aggregated data in a historical patient data storage.

20. The data processing system of claim 15, generating the outcome output display comprises generating an action tree based on the determined next action in a treatment regime using the RL model with no distance function, optimal next action in the treatment regime with allowed deviation from the guidelines, and next action in the treatment regime that adheres to the guidelines.
